(19) United States
(12) Patent Application Publication
(10) Pub. No.: US 2024/0310012 A1
(43) Pub. Date: Sep. 19, 2024

(54) A HYBRID DISINFECTION LIGHTING DEVICE

(71) Applicant: SIGNIFY HOLDING B.V., EINDHOVEN (NL)

(72) Inventors: TOBIAS BORRA, RIJSWIJK (NL); DZMITRY VIKTOROVICH ALIAKSEYEU, EINDHOVEN (NL); KARS-MICHIEL HUBERT LENSSEN, VELDHOVEN (NL); BARTEL MARINUS VAN DE SLUIS, EINDHOVEN (NL)

(21) Appl. No.: 18/579,554

(22) PCT Filed: Jul. 7, 2022
(86) PCT No.: PCT/EP2022/068868

§ 371 Date: Jan. 16, 2024

(30) Foreign Application Priority Data
Jul. 16, 2021 (EP) 21186077.0

(51) Int. Cl. F21S 8/04; A61L 2/10; G02B 27/30

(52) U.S. Cl. CPC F21S 8/04; A61L 2/10; G02B 27/30; A61L 2202/11; A61L 2202/25

(57) ABSTRACT

The invention relates to a luminaire (100) that can simultaneously be used for disinfection and for illumination. The luminaire (100) comprises a light engine (120) located in a housing, and an optical component (130) for receiving a light output of the light engine (120). The light engine (120) has a first light source (121) for emitting visible light and a second light source (122) for emitting ultraviolet radiation. The input side (131), the output side (132), and the wall structure (133) together delimit an interior volume of the optical component (130). The input side (131), the output side (132) and the interior volume of the optical component (130) are transmissive for visible light and for ultraviolet radiation. The wall structure (133) of the optical component (130) is transmissive for visible light and non-transmissive for ultraviolet radiation. The optical component (130) has a wall structure (133) separating an input side (131) and an output side (132). The input side (131), the output side (132) and an interior volume of the optical component (130) are transmissive for visible light and for ultraviolet radiation, while the wall structure (133) of the optical component (130) is transmissive for visible light and non-transmissive for ultraviolet radiation.

### FIELD OF THE INVENTION

The invention relates to a hybrid disinfection lighting device. In particular, the invention relates to a luminaire that can simultaneously be used for disinfection and for illumination.

### BACKGROUND OF THE INVENTION

Being able to prevent the spreading of infectious diseases could potentially save lives. Washing hands is a viable solution, but being able to disinfect areas with ultraviolet radiation, while minimizing risk for people, could potentially be very worthwhile.

Ultraviolet (UV) radiation is a form of electromagnetic radiation with wavelengths in a range from about 10 nm to about 400 nm.

The electromagnetic spectrum of ultraviolet radiation can be subdivided into different ranges. Three such ranges are the Ultraviolet A, B and C ranges. The Ultraviolet A (UV-A) range corresponds to radiation with wavelengths in a range of 315 to 400 nm, the Ultraviolet B (UV-B) range corresponds to radiation with wavelengths in a range of 280 to 315 nm, and the Ultraviolet C (UV-C) range corresponds to radiation with wavelengths in a range of 100 to 280 nm.

Ultraviolet radiation may be hazardous to living organisms, such as human beings. For example, skin exposure to UV-C radiation can cause erythema (sunburn-like effects). Furthermore, exposure of the eyes to UV-C radiation can produce eye injuries (photokeratitis) and (temporary) vision impairment.

Ultraviolet radiation can be used for disinfection. For example, UV-C radiation can be used in a method called “ultraviolet germicidal irradiation” (UVGI) to kill or inactivate microorganisms by destroying nucleic acids and disrupting their DNA, leaving them unable to perform vital cellular functions.

UVGI can be used to disinfect a meeting room, where the air in the room and the surface of the meeting table should be continuously cleaned by means of ultraviolet radiation, while the dose of ultraviolet radiation to which people around the table are exposed should be limited within the appropriate safety guidelines, such as those provided by the American Conference of Governmental Industrial Hygienists (ACGIH).

Although people can be exposed to a significant dose of UVC radiation without safety risks, UVC radiation is typically used in unoccupied spaces. When used in spaces where people may also be present, the UVC radiation source is typically dimmed to such a level that everywhere in the space, the irradiation at a certain height is below the safety limits for human exposure. However, this also results in a significant reduction of the irradiation that disinfects surfaces and air, even when no people can be present at a certain location, such as in the middle of a meeting table. In such a situation, it is desirable that the UV irradiation at the table surface is optimal, while people sitting around the table are exposed within lower, safe limits. This issue may be even more relevant when a distinction is made between the allowed UV radiation doses for skin and for eyes. In the middle of the table, the limit for skin is applicable, while around the table the lower limit for eyes should be met.

In rooms where people may be present, upper-air UVGI devices can be installed to continuously disinfect air. These devices are usually installed at a height of above 2.4 meters. By making use of parabolic reflectors, and non-reflective (typically black) lamellas, the UVGI can be concentrated in a zone. In this zone a high intensity UVC radiation is realized without having too high intensities below 2.0 meters. Because of the presence of natural or mechanical ventilation in the room, the contaminated room-air will pass through the UVC zone and thus will be disinfected. Upper-air UVGI devices may have an added functionality of also being able to provide ambient visible lighting.

### SUMMARY OF THE INVENTION

It is an object of the invention to provide a luminaire that can simultaneously be used for disinfection and for illumination, wherein the efficiency of disinfection is maximized while exposure of human beings to potentially hazardous radiation is minimized.

According to an aspect of the invention, the aforementioned object is achieved by a luminaire comprising (i) a housing having a light exit window, (ii) a light engine for emitting a light output, the light engine being located in the housing, and (iii) an optical component for receiving the light output, the optical component being provided at the light exit window such that at least a part of the light exit window is covered by the optical component. The optical component has an input side facing the light engine, an output side facing away from the light engine, and a wall structure separating the input side and the output side. The light engine comprises a first light source for emitting a first light output component and a second light source for emitting a second light output component, the first light output component and the second light output component together representing the light output of the light engine. The first light output component comprises visible light and the second light output component comprises ultraviolet radiation. The input side, the output side, and the wall structure together delimit an interior volume of the optical component. The input side, the output side and the interior volume of the optical component are transmissive for visible light and for ultraviolet radiation. The wall structure of the optical component is transmissive for visible light and non-transmissive for ultraviolet radiation.

The term “transmissive” refers to a material that allows electromagnetic radiation (such as visible light and ultraviolet radiation) to pass through. The term “transmissive” includes terms such as “transparent” and “translucent”. A “transparent” material allows radiation to pass through without appreciable scattering of the radiation. A “translucent” material also allows radiation to pass through, but the photons may be scattered at an interface and/or internally, for example where there is a change in index of refraction. A material that is transmissive for visible light allows at least some visible light to pass through, but not necessarily all wavelengths of visible light. A “non-transmissive” material does not allow electromagnetic radiation (such as visible light and ultraviolet radiation) to pass through. Instead, it may reflect, scatter, or absorb such radiation. Such a material may also be referred to as an opaque material.

As already mentioned in the background section, the term “ultraviolet radiation” refers to electromagnetic radiation with wavelengths in a range from about 10 nm to about 400 nm. Ultraviolet radiation may be hazardous to living organisms, but it can be used for disinfection purposes.

The term “visible light” refers to electromagnetic radiation within the portion of the electromagnetic spectrum that can be perceived by the human visual system. The human visual system responds only to radiation in a very narrow band of the electromagnetic spectrum. This range of wavelengths is approximately from 380 to 780 nanometers, depending on the individual observer. Visible light can be used for many different purposes, such as for illumination.

The luminaire according to the invention may be positioned relative to an object so as to emit directional ultraviolet radiation to the object, while limiting the ultraviolet radiation towards human beings approaching or surrounding the object. The luminaire is at the same time arranged to emit visible light for illuminating both the object and the surrounding area.

The optical component may comprise a plurality of cells arranged in a grid (or array). Each cell has an input window, an output window, and a cell wall separating the input window and the output window. The input windows together constitute the input side of the optical component, the output windows together constitute the output side of the optical component, and the cell walls together constitute the wall structure of the optical component.

The term “grid” refers to an arrangement of geometric shapes (such as the cells of the optical component), wherein the arrangement may have a repeating pattern (which may also be referred to as a periodic tiling) or a non-repeating pattern (which may also be referred to as a non-periodic tiling). The geometric shapes that are arranged in a grid may all have the same shape, but they may also have different shapes.

The cells of the above optical component may be hexagonal prismatic cells or cylindrical cells. A hexagonal prismatic cell is a cell with a shape in the form of a hexagonal prism, being a prism with a hexagonal base. When the cells of the optical component are hexagonal prismatic cells, the optical component has a honeycomb structure. A cylindrical cell is a cell with a shape in the form of a cylinder. The cells of the above optical component may have any suitable shape that allows the cells to be arranged in a grid.

The wall structure of the optical component is non-transmissive for ultraviolet radiation. To achieve this, the wall structure may be capable of reflecting, scattering, and/or absorbing ultraviolet radiation. For this purpose, the wall structure of the optical component may comprise a material chosen from the group consisting of glass and polycarbonate. If the wall structure comprises glass, it may have a bilayer coating of titanium dioxide and zinc oxide on a glass substrate. Such a coating can effectively absorb ultraviolet radiation while allowing visible light to pass through.

The light engine may further comprise a collimator for collimating the second light output component. When the second light output component is collimated before it reaches the optical component, it is easier to provide the desired directional ultraviolet radiation output.

The first light source of the light engine may comprise an edge-lit light guide panel. This ensures that the first light output component is distributed substantially homogenously over the light exit window. It may also allow the first and second light sources of the light engine to be stacked on top of each other, wherein the second light source can be arranged such as to emit the second light output component through the light guide panel of the first light source towards the optical component, provided of course that the light guide panel is made from a material that is transmissive for ultraviolet radiation.

The wall structure of the optical component may have a first wall structure part adjacent to the input side and a second wall structure part adjacent to the output side, wherein the first wall structure part is capable of reflecting ultraviolet radiation and the second wall structure part is capable of absorbing ultraviolet radiation. This configuration allows to control the size of the ultraviolet radiation beam and to optimize for ultraviolet energy transfer.

The wall structure of the optical component may have a coating of a photoluminescent material capable of converting the ultraviolet radiation of the second light output into visible light. This configuration allows the efficiency of the luminaire to be improved and/or the need for a separate source of visible light next to a source for ultraviolet radiation to be eliminated, thereby saving costs and complexity.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1(a) and 1 (b) show a luminaire in cross-sectional view.

FIG. 2 shows an optical component.

FIGS. 3(a) and 3(b) show a cell of an optical component.

FIGS. 4(a) and 4(b) show a luminaire in cross-sectional view.

FIGS. 5(a) and 5(b) show a luminaire in cross-sectional view.

FIG. 6 shows a cell of an optical component.

The schematic drawings are not necessarily to scale.

### DETAILED DESCRIPTION OF THE EMBODIMENTS

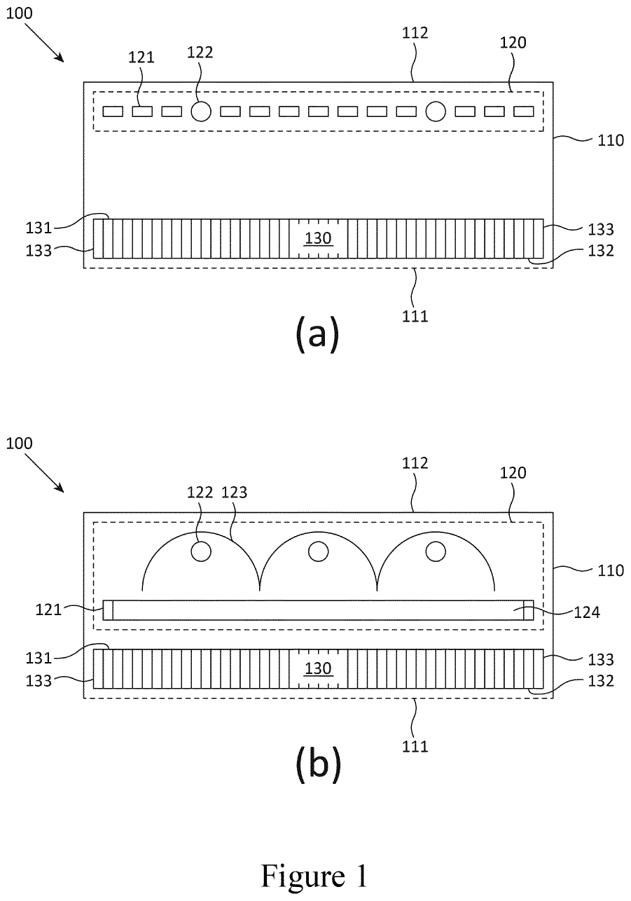

Each of FIGS. 1(a) and 1(b) shows a luminaire 100 in cross-sectional view.

The luminaire 100 of FIG. 1(a) has a housing 110, wherein the housing 110 has a light exit window 111. The housing 110 further has a back panel 112, located opposite from the light exit window 111, and side walls (not numbered). The back panel 112 may have a means for mounting the luminaire 100 to a mounting surface, such as a ceiling.

The luminaire 100 further has a light engine 120. The light engine 120 is located in the housing 110, and it is arranged to emit a light output. The light engine 120 comprises a first light source 121 and a second light source 122. The first light source 121 is arranged to emit a first light output component, and the second light source 122 is arranged to emit a second light output component. The first light output component and the second light output component together represent the light output of the light engine 120.

The first light output component emitted by the first light source 121 comprises visible light. The second light output component emitted by the second light source 122 comprises ultraviolet radiation.

The visible light of the first light output component may be used for illumination, while the ultraviolet radiation of the second light output component may be used for disinfection.

In FIG. 1(a), the first light source 121 comprises a plurality of light-emitting diodes, and the second light source 122 comprises a plurality of UV-tubes. The UV-tubes of the second light source 122 are located in between the light emitting diodes of the first light source 121. For the purpose of the invention, the first light source may be of any suitable type, as long as it is capable of emitting visible light. Also, the second light source may be of any suitable type, as long as it is capable of emitting ultraviolet radiation.

The luminaire 100 further has an optical component 130. The optical component 130 is being provided at the light exit window 111 of the housing 110. The optical component 130 covers substantially all of the light exit window 111. Alternatively, the optical component may only cover a part of the light exit window. The optical component 130 is arranged to receive the light output of the light engine 120.

The optical component 130 has an input side 131 facing the light engine 120 and an output side 132 facing away from the light engine 120. The optical component 130 further has a wall structure 133 separating the input side 131 and the output side 132.

The input side 131, the output side 132, and the wall structure 133 together delimit an interior volume of the optical component 130. The input side 131, the output side 132 and the interior volume of the optical component 130 are transmissive for visible light (provided by the first light source 121) and for ultraviolet radiation (provided by the second light source 122). The wall structure 133 of the optical component 130 is also transmissive for visible light (provided by the first light source 121), but it is non-transmissive for ultraviolet radiation (provided by the second light source 122). The wall structure 133 can be non-transmissive for ultraviolet radiation because it is arranged to absorb and/or reflect ultraviolet radiation. In other words, the wall structure 133 may be made from an opaque material.

The luminaire 100 of FIG. 1(b) is similar to the one illustrated in FIG. 1(a), but now the light engine 120 has a first light source 121 that comprises an edge-lit light guide panel 124. The first light source 121 is located between the second light source 122 and the optical component 130. The second light source 122 comprises a plurality of UV-tubes, and each of these UV-tubes is provided with a parabolic mirror. The parabolic mirrors together form a collimator 123, wherein the collimator 123 is a component of the light engine 120 and arranged to collimate the second light output component of the second light source 122. The second light output component of the second light source 122 is incident on the edge-lit light guide panel 124. The latter is transmissive for the second light output component so that the second light output component can be received by the optical component 130.

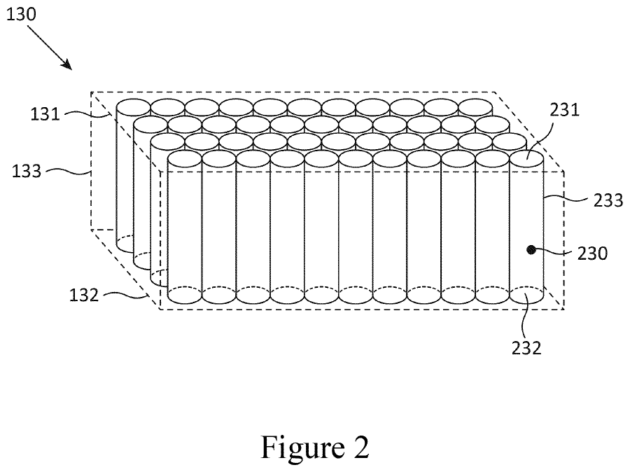

FIG. 2 shows an optical component 130 that comprises a plurality of cells 230 arranged in a grid. The cells 230 are cylindrical cells, but they may instead also be hexagonal prismatic cells, or any other suitable type of cells.

Each cell 230 has an input window 231, an output window 232, and a cell wall 233 separating the input window 231 and the output window 232. The input windows 231 together constitute the input side 131 of the optical component 130, the output windows 232 together constitute the output side 132 of the optical component 130, and the cell walls 233 together constitute the wall structure 133 of the optical component 130.

For each cell 230, the input window 231, the output window 232, and the cell wall 233 delimit an interior volume of the cell 230.

For each cell 230, the input window 231, the output window 232, the cell wall 233 and the interior volume of the cell 230 are transmissive for visible light. Consequently, the input side 131, the output side 132, the wall structure 133 and the interior volume of the optical component 130 are transmissive for visible light.

For each cell 230, the input window 231, the output window 232, and the interior volume of the cell 230 are also transmissive for ultraviolet radiation. Consequently, the input side 131, the output side 132, and the interior volume of the optical component 130 are transmissive for ultraviolet radiation.

For each cell 230, the cell wall 233 is non-transmissive for ultraviolet radiation. Consequently, the wall structure 133 of the optical component 130 is non-transmissive for ultraviolet radiation.

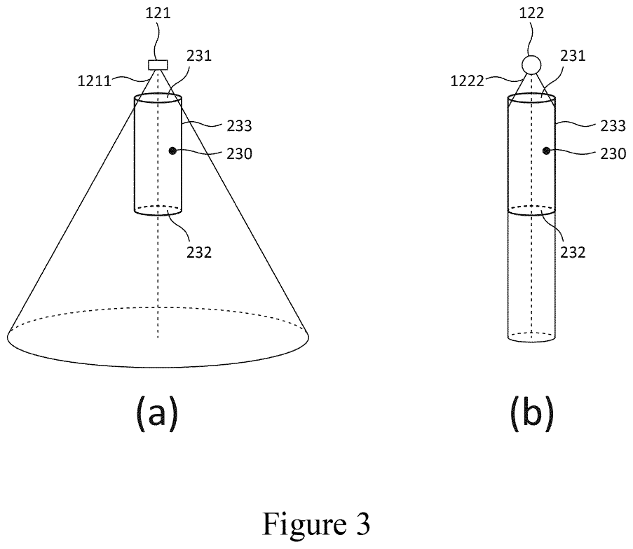

Each of FIGS. 3(a) and 3(b) shows a cell 230 of the optical component 130. The cell 230 has an input window 231, an output window 232, and a cell wall 233 separating the input window 231 and the output window 232.

In FIG. 3(a), a first light source 121 emits a first light output component 1211, which comprises visible light. The first light output component 1211 is incident on the input window 231. Because the input window 231 is transmissive for visible light, the first light output component 1211 passes through the input window 231 into the interior of the cell 230. Via the interior of the cell 230, which is also transmissive for visible light, the first light output component 1211 is incident on the cell wall 233 and on the output window 232. Because each of the cell wall 233 and the output window 232 is also transmissive for visible light, the first light output component 1211 passes through the cell wall 233 and through the output window 232, and hence out of the cell 230.

In FIG. 3(b), a second light source 122 emits a second light output component 1222, which comprises ultraviolet radiation. The second light output component 1222 is incident on the input window 231. Because the input window 231 is transmissive for ultraviolet radiation, the second light output component 1222 passes through the input window 231 into the interior of the cell 230. Via the interior of the cell 230, which is also transmissive for ultraviolet radiation, the second light output component 1222 is incident on the cell wall 233 and on the output window 232. Because the output window 232 is also transmissive for ultraviolet radiation, the second light output component 1222 passes through the output window 232, and hence out of the cell 230. However, because the cell wall 233 is non-transmissive for ultraviolet radiation, the second light output component 1222 cannot pass through the cell wall 233 to leave the cell 230.

Due to the configuration of the cell 230 as described above, the angular distribution of the first light output component 1211 is not (or only slightly) influenced by the cell 230. In contrast to this, the angular distribution of the second light output component 1222 is influenced by the cell 230, such that upon leaving the cell 230, the second light output component 1222 is collimated or narrowed.

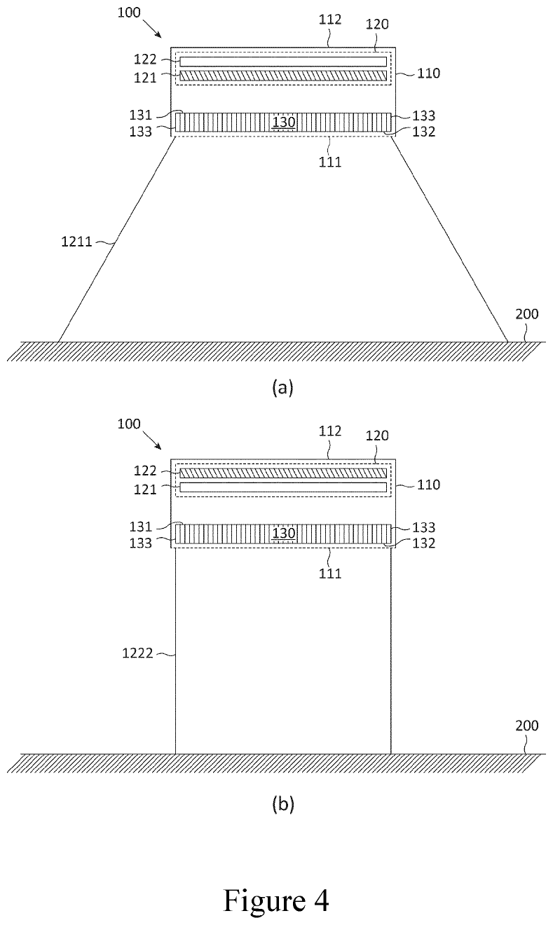

Each of FIGS. 4(a) and 4(b) show a luminaire 100 as already illustrated in FIG. 1. For the sake of simplicity, the first light source 121 and the second light source 122 are here indicated by rectangles that are located on top of each other. However, it should be understood that each of the configurations illustrated in Figures l(a) and 1(b) can be used, or any other suitable arrangement of the first light source 121 and the second light source 122.

In FIG. 4(a), the first light source 121 is switched on and emits a first light output component 1211. The first light output component 1211 passes through the optical component towards a target surface 200, with no (or almost no) change in the angular distribution.

In FIG. 4(b), the second light source 122 is switched on and emits a second light output component 1222. The second light output component 1222 passes through the optical component towards a target surface 200, with a change in the angular distribution so that the second light output component 1222 is collimated or narrowed.

When the first light source 121 and the second light source 122 are both switched on, the first light output component 1211 and the second light output component 1222 together represent the light output of the light engine 120. The light output of the light engine 120 then has visible light with a relatively broad angular distribution as shown in FIG. 4(a) and ultraviolet radiation with a relatively narrow angular distribution as shown in FIG. 4(b).

The luminaire 100 as described above, represents a hybrid disinfection lighting device. It may be positioned above frequently used or contaminated objects, such as a desk, a counter, a washing stand, and a toilet. The luminaire 100 is then arranged to emit directional ultraviolet radiation to the object below, while limiting the ultraviolet radiation towards human beings approaching or surrounding the object below. The luminaire 100 is at the same time arranged to emit visible light for properly illuminating both the object below and the surrounding area.

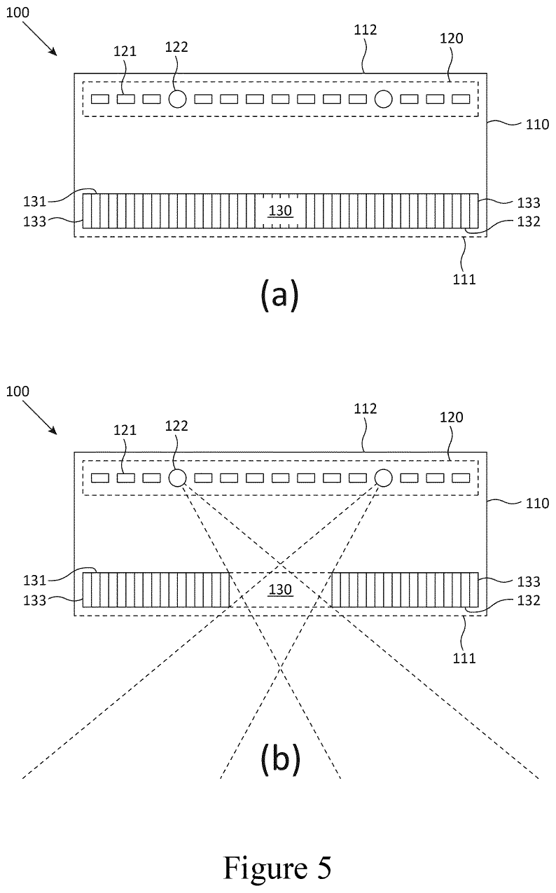

FIG. 5(a) shows the same luminaire 100 as already shown in FIG. 1(a). FIG. 5(b) shows a luminaire 100 wherein the optical component 130 has an opening in the center so that the optical component 130 covers only part of the light exit window 111. The opening in the center of the optical component 130 is delimited by the input side 131, by the output side 132, and by one or more side walls that are part of the wall structure 133. Such an opening in the center of the optical component 130, or any other configuration wherein the optical component covers only part of the light exit window 111, still provides a collimated or narrowed second light output component 1222, irrespective of the relative arrangement of the second light source 122 and the optical component 130. However, the relative arrangement of the second light source 122 and the optical component 130 may be such that the optical component 130 prevents a direct view of the second light source 122 from typical viewing angles. This would be the case for the luminaire 100 of FIG. 5(b), where the dashed lines originating from the second light source 122 indicate the angular range wherein an observer may have a direct view of the second light source. A direct view of the second light source 122 is not preferred, because the second light source 122 emits potentially harmful ultraviolet radiation.

Because the purpose of the optical component is to collimate or narrow the angular distribution of the second light output component leaving the luminaire, the optical component is preferably arranged to intercept most, if not all, of the second light output component. In case the second light output component is directly incident on only a part of the light exit window, then only that part of the light exit window may be covered by the optical component.

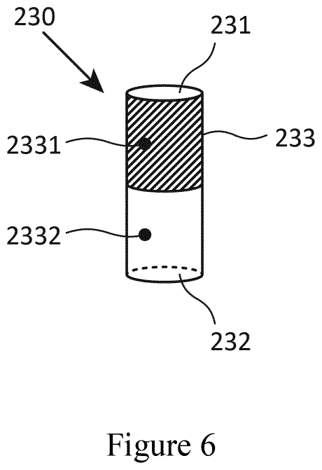

FIG. 6 shows a cell 230 of the optical component 130. The cell 230 has an input window 231, an output window 232, and a cell wall 233 separating the input window 231 and the output window 232. The optical component 130 comprises a plurality of cells 230, of which the input windows 231 together form the input side 231 of the optical component 130, the output windows 232 together form the output side 232 of the optical component 130, and the cell walls 233 together form the wall structure 133 of the optical component 130.

For each cell 230, the cell wall 233 has a first cell wall part 2331 adjacent to the input window 231 and a second cell wall part adjacent to the output window 232. The first cell wall parts 2331 together form a first wall structure part of the wall structure 133 of the optical component 130, and the second cell wall parts 2332 together form a second wall structure part of the wall structure 133 of the optical component 130.

The first cell wall part 2331 is capable of reflecting ultraviolet radiation. The second cell wall part 2332 is capable of absorbing ultraviolet radiation. Consequently, the first wall structure part of the optical component 130 is capable of reflecting at least a portion of the second light output component, while the second wall structure part of the optical component 130 is capable of absorbing at least a portion of the second light output component.

Using materials with different reflectivity and absorption in different sub-ranges of the ultraviolet radiation spectrum may be used to control the size of the ultraviolet radiation beam and to optimize for ultraviolet energy transfer. For the cells 230 as illustrated in FIG. 6, the first cell wall part 2331 is arranged to reduce loss, while the second cell wall part 2332 is arranged to ensure that any ultraviolet radiation that is not parallel to the cell wall 233 is absorbed.

Materials capable of reflecting ultraviolet radiation and transmitting visible light are known in the art. For example, a material comprising periodic porous multilayers with photonic crystal properties can be made by spin-coating-assisted layer-by-layer deposition of colloidal suspensions of nanoparticles of zirconium dioxide and silicon dioxide to provide an optical filter that can reflect well-defined wavelength ranges of UV-A, UV-B, and UV-C radiation while preserving transmissivity for visible radiation.

Materials capable of absorbing ultraviolet radiation and transmitting visible light are also known in the art. For example, a bilayer porous film structure may be used to absorb ultraviolet radiation with relatively high transmissivity for visible light. To effectively absorb UV-B and UV-A radiation, titanium dioxide and zinc oxide can be used as absorbing layers.

In an alternative configuration, the wall structure of the optical component may comprise a coating of a photoluminescent material that is capable of converting ultraviolet radiation into visible light. Suitable examples of such photoluminescent materials are phosphors similar to those used in fluorescent light tubes. In this way, the efficiency of the luminaire can be improved and/or the need for a separate source of visible light next to a source for ultraviolet radiation can be eliminated, thereby saving costs and complexity.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

The various aspects discussed in this patent can be combined in order to provide additional advantages. Further, the person skilled in the art will understand that embodiments can be combined, and that also more than two embodiments can be combined.

1. A luminaire comprising:
a housing having a light exit window,
a light engine for emitting a light output, the light engine being located in the housing, and
an optical component for receiving the light output, the optical component being provided at the light exit window such that at least a part of the light exit window is covered by the optical component,
wherein the optical component has an input side facing the light engine, an output side facing away from the light engine, and a wall structure separating the input side and the output side,
wherein the light engine comprises a first light source for emitting a first light output component and a second light source for emitting a second light output component, the first light output component and the second light output component together representing the light output of the light engine,
wherein the first light output component comprises visible light and the second light output component comprises ultraviolet radiation,
wherein the input side, the output side, and the wall structure together delimit an interior volume of the optical component,
wherein the input side, the output side and the interior volume of the optical component are transmissive for visible light and for ultraviolet radiation, and
wherein the wall structure of the optical component is transmissive for visible light and non-transmissive for ultraviolet radiation.

2. A luminaire according to claim 1, wherein the optical component comprises a plurality of cells arranged in a grid, each cell having an input window, an output window, and a cell wall separating the input window and the output window, the input windows together constituting the input side of the optical component, the output windows together constituting the output side of the optical component, and the cell walls together constituting the wall structure of the optical component.

3. A luminaire according to claim 2, wherein the cells are hexagonal prismatic cells or cylindrical cells.

4. A luminaire according to claim 1, wherein the wall structure of the optical component is capable of absorbing ultraviolet radiation.

5. A luminaire according to claim 1, wherein the wall structure of the optical component comprises glass or polycarbonate.

6. A luminaire according to claim 5, wherein the wall structure of the optical component comprises a glass substrate having a bilayer coating of titanium dioxide and zinc oxide.

7. A luminaire according to claim 1, wherein the light engine further comprises a collimator for collimating the second light output component.

8. A luminaire according to claim 1, wherein the first light source comprises an edge-lit light guide panel.

9. A luminaire according to claim 1, wherein the wall structure of the optical component has a first wall structure part adjacent to the input side and a second wall structure part adjacent to the output side, the first wall structure part being capable of reflecting ultraviolet radiation and the second wall structure part being capable of absorbing ultraviolet radiation.

10. A luminaire according to claim 1, wherein the wall structure of the optical component has a coating of a photoluminescent material capable of converting the ultraviolet radiation of the second light output into visible light.
